(19) United States
(12) Patent Application Publication
(10) Pub. No.: US 2011/0224536 A1
(43) Pub. Date: Sep. 15, 2011

(54) SYSTEMS AND METHODS FOR DETERMINING A TEMPERATURE DIFFERENTIAL USING TEMPERATURE SENSITIVE MAGNETIC RESONANCE IMAGING

(75) Inventors: John Pile-Spellman, Pelham, NY (US); Erwin Lin, Whitestone, NY (US)

(73) Assignee: THE TRUSTEES OF COLUMBIA UNIVERSITY IN THE CITY OF NEW YORK, New York, NY (US)

(21) Appl. No.: 13/056,019

(22) PCT Filed: Aug. 1, 2008
(86) PCT No.: PCT/US08/71995

§ 371 Date: May 24, 2011

(51) Int. Cl. A61B 5/055

(52) U.S. Cl. 600/412

(57) ABSTRACT

A method and apparatus for determining a temperature differential at a portion of a patient's body utilizing temperature sensitive MRI measurements. A diagnostic fluid bolus is administered into a blood vessel of the patient, wherein the diagnostic fluid bolus has a diagnostic fluid bolus temperature waveform. MRI measurements are used to determine the thermodiluted temperature waveform of the diagnostic fluid bolus at a target site in the body spaced away from the administration site. The temperature differential may be used to determine a cardiovascular parameter.

### CROSS REFERENCE TO RELATED APPLICATIONS

This application is a 371 of International Patent Application No. PCT/US2008/071995, filed Aug. 1, 2008, the contents which are incorporated herein by reference in their entirety.

### FIELD OF THE INVENTION

The present invention relates to systems and methods for determining a cardiovascular parameter based on a temperature differential determined from information obtained by magnetic resonance imaging.

### BACKGROUND

Tissue perfusion is a measure of the delivery of blood to a part of the body. While perfusion to an organ can be viewed on a global level, such as perfusion to an entire organ, perfusion can also be viewed on a local level, such as perfusion to a small region. Many disease processes cause perfusion abnormalities at a global or local level and measurement of absolute and relative values of tissue perfusion have been used to diagnose disease and to assess the stage, degree and reversibility of disease. Non-invasive methods to measure tissue perfusion include magnetic resonance imaging (“MRI”), computerized tomography (“CT”), ultrasound (“US”) and nuclear medicine

These non-invasive methods rely primarily on dilution of an indicator or tracer introduced into a blood vessel. Specifically, a substance is introduced into the cardiovascular system and the concentration of the indicator in a voxel or a larger region is measured to calculate parameters that reflect relative or absolute measures of tissue perfusion. The concentration of an indicator within a voxel is determined by the quantity of indicator delivered to the voxel, the transit time of the indicator through the voxel and the volume of distribution of the indicator within the voxel.

Indicators may be diffusible or non-diffusible based on their physical properties as well as the physical characteristics of the vessels and tissue being perfused. Non-diffusible indicators, such as gadolinium contrast agents used in the brain, remain confined to blood vessels and their concentration is therefore dependent on the volume of blood vessels (i.e., the “blood volume”) within the voxel. Diffusible indicators, such as gadolinium contrast agents used outside of the central nervous system or labeled protons using arterial spin labeling, can freely diffuse into the voxel interstitium and their concentration is therefore determined by the sum of the blood volume and the interstitial volume of the voxel.

Whether using a currently available non-diffusible or diffusible indicator, a variety of assumptions and estimations may have to be made when using MRI to measure tissue perfusion. Specifically, assumptions may have to be made to calculate tissue concentration from MR signal or phase change measurements. For example, when using gadolinium contrast agents in the brain, assuming T1 effects can be ignored results in a linear relationship between local tissue concentration of gadolinium and changes in T2 relaxation. Assumptions and estimations are a potential source of error when the calculated tissue concentrations are then used to calculate cardiovascular parameters such as flow, volume of distribution and mean transit time. When using arterial spin labeling, calculations used to obtain tissue concentration of labeled spins based on MR signal measurements require complex alterations of the Bloch equations. Furthermore, unless the arterial input function is known, such as by using an intra-arterial injection of indicator through a catheter, or measured in a major artery supplying the tissue of interest, only relative values of the flow to volume ratio may be calculated, regardless of the technique utilized.

When using gadolinium-based techniques, only a single dose of gadolinium contrast agent can typically be administered at any one time due to safety concerns. In addition, gadolinium contrast agents are expensive.

A need therefore exists for a MRI method and system for measuring perfusion using a diffusible indicator that has more ideal properties and allows simpler and more accurate calculations.

Reference throughout this specification to “an embodiment” or “an exemplary embodiment” means that a particular feature, structure, or characteristic described in connection with the embodiment is included in at least one embodiment of the present invention. Thus, the appearances of these phrases in various places throughout this specification are not necessarily all referring to the same embodiment. Furthermore, the particular features, structures, or characteristics may be combined in any suitable manner in one or more embodiments.

### SUMMARY

In one aspect, the present invention provides method for determining a temperature differential in a portion of a patient's body, comprising administering a diagnostic fluid bolus into a blood vessel of the patient, wherein the temperature waveform of the diagnostic fluid bolus is at least one of: i) a waveform having an edge-enhancing feature; and ii) selected using information obtained from a test fluid bolus administered into the blood vessel after the test fluid bolus has traveled a distance to the portion of the body. A diagnostic set of magnetic resonance information is obtained from the portion of the body. A magnetic resonance parameter from the portion of the body is determined using the diagnostic set of magnetic resonance information. A temperature differential in the portion of the body is determined using the magnetic resonance parameter.

The diagnostic fluid bolus may be selected using a test fluid bolus into the blood vessel of the patient, wherein the test fluid bolus has a temperature waveform. A test set of magnetic resonance information may be obtained from the portion of the body. The test set of magnetic resonance information may be used to determine a thermodiluted temperature waveform that results from thermodilution of the test fluid bolus as the test fluid bolus travels to the portion of the body. A deconvolution function may be determined that transforms the thermodiluted temperature waveform into the temperature waveform of the test fluid bolus. The deconvolution function may be applied to the temperature waveform of the test fluid bolus to obtain the temperature waveform for the diagnostic fluid bolus.

In another aspect, the present invention provides an apparatus for determining a temperature differential in a portion of a patient's body, the apparatus comprising: a fluid source (which may have a liquid or gas as the fluid); a fluid control system adapted to control flow of the fluid from the fluid source into the patient; and a controller in communication with the fluid control system. The controller may be adapted to operate the fluid control system to deliver a diagnostic fluid bolus to be administered into a blood vessel of the patient, wherein the temperature waveform of the diagnostic fluid bolus is at least one of: i) a waveform having an edge-enhancing feature; and ii) selected using information obtained from a test fluid bolus administered into the blood vessel after the test fluid bolus has traveled a distance to the portion of the body.

The controller may also obtain a diagnostic set of magnetic resonance information from a portion of the patient's body after administration of the diagnostic fluid bolus. The controller may also determine a magnetic resonance parameter from the portion of the body using the diagnostic set magnetic resonance information and determine a temperature differential in the portion of the body using the magnetic resonance parameter. The fluid control system may include one or more pumps and/or one or more valves.

### BRIEF DESCRIPTION OF THE DRAWINGS

The present invention will become more fully understood from the detailed description given herein below and the accompanying drawings which are given by way of illustration only and wherein:

FIG. 1 is a flow diagram that illustrates an embodiment of a method of measuring a cardiovascular parameter using temperature sensitive MRI.

FIG. 2 depicts an embodiment of a system for controlling the temperature of a fluid that is introduced into a patient.

FIG. 3 is a block diagram that depicts an embodiment of a user computing device

FIG. 4 is a block diagram that depicts an embodiment of a network architecture.

FIG. 5 is a graph of temperature changes in a capillary phantom as a function of time, calculated according to an embodiment of the invention, using sequential dynamic phase images following an injection of a cold saline bolus. Temperature change with respect to baseline (room temperature) is shown on the vertical axis in units of degrees Centigrade. Time, represented by image number (where the time between images is a fixed constant) is shown on the horizontal axis increasing from left to right.

FIG. 6 is a graph showing the measured temperature as a function of time at a thermometer 1 (A) and a thermometer 2 (B) that corresponds to the cold saline bolus of FIG. 5. The baseline temperature is slightly greater than 21° C.

FIG. 7 is a graph of calculated temperature changes in a capillary phantom as determined by sequential dynamic phase images as a function of time following an injection of a room temperature saline bolus. Temperature change with respect to baseline (room temperature) is shown on the vertical axis in units of degrees Centigrade. Time, represented by image number (where the time between images is a fixed constant) is shown on the horizontal axis increasing from left to right.

FIG. 8 is a graph showing the measured temperature as a function of time at a thermometer 1 (A) and a thermometer 2 (B) that corresponds to the room temperature bolus of FIG. 7. The baseline temperature is slightly greater than 21° C.

FIGS. 9A-9D show temperature waveforms of fluid boluses and their subsequent thermodilution at various stages during the performance of a method according to an exemplary embodiment of the present invention.

FIG. 10 shows an apparatus for delivering a fluid bolus according to an exemplary embodiment of the present invention.

### DETAILED DESCRIPTION OF THE INVENTION

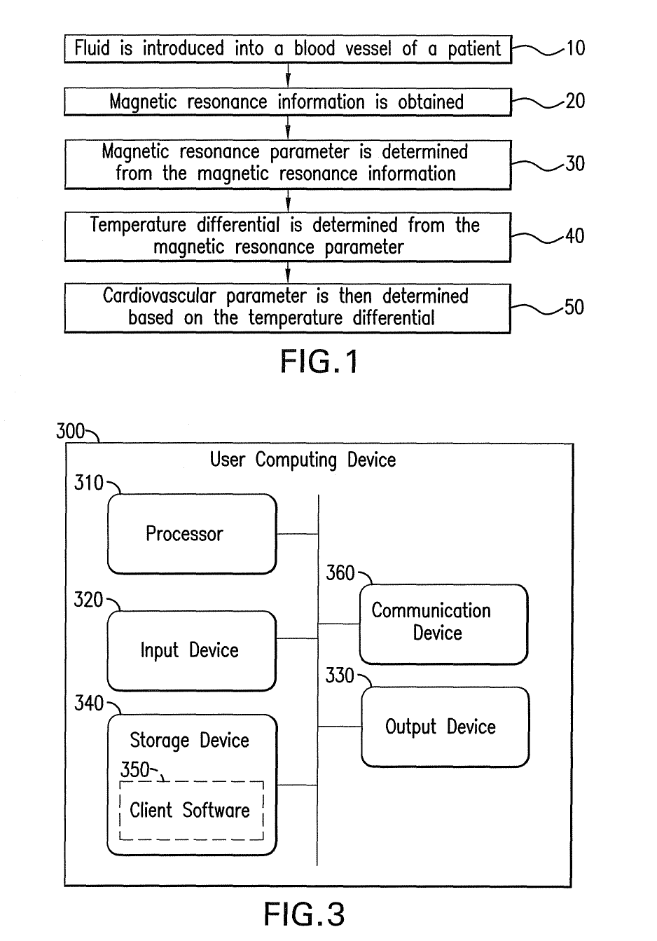

In an embodiment, the present invention provides a method for determining a cardiovascular parameter in a portion of a body of a patient based on a temperature differential of the portion of the body determined from information obtained by MRI. Specifically, referring to FIG. 1, a method for determining a cardiovascular parameter comprises introducing a fluid into a blood vessel of a patient (10) and then obtaining magnetic resonance information from a portion of the body of the patient (20). A magnetic resonance parameter is determined using the magnetic resonance information (30) and a temperature differential in the portion of the body is determined using the magnetic resonance parameter (40). Based on the temperature differential, a cardiovascular parameter is determined (50).

A cardiovascular parameter that is determined in a portion of the body can be any cardiovascular parameter (qualitative and/or quantitative) associated with tissue perfusion. Non-limiting examples of cardiovascular parameters are volume of distribution, blood flow, transit time including mean transit time, and any combination thereof. Volume of distribution is the volume of tissue in the portion of the body in which heat is distributed. Blood flow is the volume of blood moving through the portion of the body per unit time. Transit time is the time required for an individual fluid molecule to flow through the volume of distribution from an arterial input to a venous output. Mean transit time is a bulk property of the fluid and is the average time required for individual fluid molecules to flow through a given region of the part of the body from an arterial input to a venous output. Methods of the present invention include determining a single cardiovascular parameter or multiple cardiovascular parameters.

The cardiovascular parameter can be for a portion of the body, such as an organ or tissue. Non-limiting examples of organs for which a cardiovascular parameter can be determined include the brain, lungs, heart, kidney, liver, stomach and other gastrointestinal organs, and vasculature. Vasculature includes arteries and veins including central and peripheral arteries and veins. For example, the artery can be the carotid artery and the vein can be an internal jugular vein or a large vein draining an organ.

Referring again to FIG. 1, with respect to introducing a fluid into a blood vessel of a patient (10), the fluid can be any biologically compatible fluid that can perfuse the portion of the body. For example, the fluid may be water, blood or a saline solution. The fluid can be introduced over any time frame at any rate sufficient to induce temperature changes that can be effectively imaged. For example, the fluid may be introduced at a constant rate over a period of seconds, such as, for example, a bolus injection where the shape of the input is a square wave. Alternatively, the fluid may be introduced over a period of minutes, where the shape of the input is a desired function of time including a sinusoidal function. Furthermore, the shape of the input may be designed to optimize the arterial input function of the blood vessel being imaged and thereby simplify calculations.

The fluid can be introduced in any manner such that the fluid can perfuse the portion of the body and induce temperature changes that can be effectively imaged. For example, the fluid can be injected intravenously or intra-arterially or introduced as a gas into the lungs via inhalation. Further, the fluid can be introduced at a site local or distant to the portion of the body in which the cardiovascular parameter is being determined. For example, the fluid may be injected into a peripheral vein using a conventional intravenous line, into a central vein using a central venous line, or through a catheter or needle in a peripheral or central artery that supplies the portion of the body in which perfusion is to be determined.

In an exemplary embodiment, the temperature of the introduced fluid can be above or below body temperature. Further, the temperature of the introduced fluid may have a uniform constant temperature below or above body temperature or can vary over time and include temperatures above and below body temperature. For example, the introduced fluid may vary over time when the injection site is remote from the tissue of interest, such as a peripheral vein, and the profile of the injected fluid changes after passing through the heart and pulmonary circulation. Using an injection with a time-varying temperature may reduce such changes. A constant temperature injection may be used, for example, when the injection site is closer to the tissue of interest, such as a central artery, and the profile of the injected fluid does not change as readily.

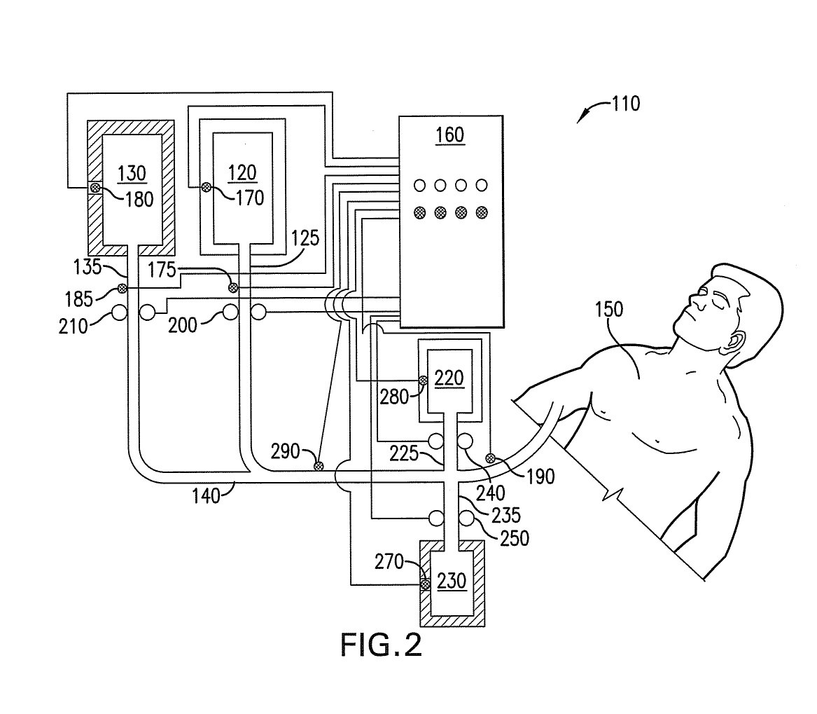

A system can be used for controlling the temperature of the fluid that is introduced into the patient by combining fluids having two different temperatures and introducing the combined fluid into the patient. Referring to FIG. 2, in an embodiment, such a system 110 includes first reservoir 120 containing a first fluid at a temperature below body temperature and second reservoir 130 containing a second fluid at a temperature above body temperature. First and second reservoirs 120 and 130 are in fluid communication with respective first and second fluid lines 125 and 135, which, in turn, are in fluid communication with a convergent line 140. First and second lines 125 and 135 can converge with convergent line 140 via a Y-connector, for example, such that the fluid outflow of reservoirs 120 and 130 is combined into a single fluid line.

System 110 further comprises first reservoir temperature sensor 170 in communication with first reservoir 120 and first line temperature sensor 175 in communication with first fluid line 125. System 110 further comprises second reservoir temperature sensor 180 in communication with second reservoir 130 and second line temperature sensor 185 in communication with second fluid line 135.

System 110 may further comprise optional third reservoir 220 containing a third fluid at a temperature below body temperature and optional fourth reservoir 230 containing a fourth fluid at a temperature above body temperature. Third and fourth reservoirs 220 and 230 are in fluid communication with respective third and fourth fluid lines 225 and 235, which, in turn, are in fluid communication with convergent line 140. Convergent line 140 is insertable into a blood vessel of a patient 150 either directly or indirectly, e,g, via a catheter attached to the distal end of convergent line 140. System 110 may further comprise third reservoir temperature sensor 280 in communication with third reservoir 220 and fourth reservoir temperature sensor 270 in communication with fourth reservoir 230. In addition, system 110 comprises convergent line temperature sensors 190 and 290.

System 110 further comprises controller 160 for controlling the flow of first, second, third (optional) and fourth (optional) fluids from respective first, second, third (optional) and fourth (optional) reservoirs 120, 130, 220, and 230. Specifically, in an embodiment, controller 160 is in communication with sensors 170, 180, 175, 185, 190, 270, 280 and 290. Controller 160 is also in communication with first pump 200, second pump 210, third pump 240 (optional) and fourth pump 250 (optional) which, in turn, are in communication with first fluid line 125, second fluid line 135, third fluid line 225 (optional) and fourth fluid line 235 (optional) respectively. A non-limiting example of first, second, third and fourth pumps 200, 210, 240 and 250 are power injectors.

In order to control the flow of first and second fluids, controller 160 receives temperature input signals from sensors 170, 180, 175, and 185 regarding the temperature of the first and second fluids and accordingly sends out a control signal to pumps 200 and 210 to adjust the flow rate of the fluids. Likewise, in order to control the flow of third and fourth fluids, controller 160 receives temperature input signals from sensors 280 and 270 regarding the temperature of the third and fourth fluids and accordingly sends out a control signal to pumps 240 and 250 to adjust the flow rate of the fluids.

Controller 160 may be computerized and the flow rate of first and second fluids exiting respective first and second reservoirs 120 and 130 can be varied, e.g., in accordance with a look-up table or an algorithm, so as to achieve a desired temperature variation of the introduced combined fluid. Temperature readings from the convergent line temperature sensors 190 and 290 can be used to confirm the expected temperature in convergent line 140 as determined from the look-up table or the algorithm. Controller 160 may introduce additional fluid from third and fourth reservoirs 220 and 230, e.g., in accordance with a look-up table or an algorithm, to make adjustments to achieve the desired temperature variation of the introduced fluid, as detailed below.

Referring back to FIG. 1, an embodiment of a method of the present invention includes obtaining magnetic resonance information from the portion of the body (20). The magnetic resonance information is determined by physical properties of the portion of the body and includes but is not limited to MR signal intensity, phase information, frequency information and any combination thereof. To obtain such magnetic resonance information, the patient is placed in a MR scanner and radiofrequency (RF) pulses are transmitted to the patient. The RF pulse sequences can be used to excite a slice, a series of slices or a volume of a part of the body. RF pulses can be applied in a dynamic fashion so that magnetic resonance information is measured dynamically, such as at multiple sequential points in time. For example, magnetic resonance information can be measured before, during and after the introduced fluid perfuses the portion of the body of the patient. The pulse sequences may include but are not limited to echo-planar, gradient echo, spoiled gradient echo and spin echo. For each slice, series of slices or volume, the magnetic resonance information can be spatially encoded by using magnetic field gradients including phase-encoding gradients and frequency-encoding gradients. Specifically, spatial encoding of the magnetic resonance information can be achieved by applying additional magnetic field gradients after excitation of tissue but before measurement of the magnetic resonance information (phase-encoding gradient) as well as during signal measurement (frequency-encoding gradient). In order to fully spatially encode a slice or volume of excited tissue, the excitation and measurement process can be repeated multiple times with different phase-encoding gradients. When performing a volume acquisition, two different phase encoding gradients can be applied in order to ultimately divide the volume into multiple slices. Spatial encoding allows calculation of the amount of magnetic resonance information emitted by small volume elements (voxels) in the excited slice or volume and therefore allows magnetic resonance information to be measured on a voxel-by-voxel basis in each slice, series of slices or volume.

The magnetic resonance information obtained in 20 is used to determine a magnetic resonance parameter in the portion of the body (30) according to an embodiment of a method of the present invention. The magnetic resonance parameter is determined by the physical properties of the portion of the body and non-limiting examples of magnetic resonance parameters includes phase changes resulting from changes in water proton resonance frequency; changes in T1 relaxation time; changes in diffusion coefficients; phase changes as determined by analysis of spectroscopic data; and any combination thereof. Methods for calculating such magnetic resonance parameters involve using well-known mathematical formulas based on the pulse sequence used and the specific parameter that is to be calculated. Methods of the present invention include measuring a single magnetic resonance parameter or multiple magnetic resonance parameters. The magnetic resonance parameter can be calculated on a voxel-by-voxel basis for each slice, series of slices or volume.

The magnetic resonance parameter calculated in 30 is used to calculate a temperature differential in the portion of the body (40). Methods for calculating a temperature differential based on the above-identified magnetic resonance parameters are well-known in the art. For example, if the magnetic resonance parameter is phase changes (ΔΦ) corresponding to changes in water proton resonance frequency, a corresponding temperature differential (ΔT) can be calculated in accordance with the equation ΔT=ΔΦ(T)/αγTEB₀, where α is a temperature dependent water chemical shift in parts per million (ppm) per C.°, γ is the gyromagnetic ratio of hydrogen, TE is the echo time and B₀ is the strength of the main magnetic field. The temperature differential (ΔT) in a volume of tissue (V) corresponds to a quantity of heat (ΔH) according to the formula ΔH=(ΔT)×(V)×(specific heat)×(specific gravity). The quantity of heat flowing through the arterial input of the part of the body can be calculated by obtaining slices through the arterial input and integrating ΔH over time.

With respect to calculating a temperature differential based on changes in T1 relaxation time, changes in diffusion coefficients, or phase changes as determined by analysis of spectroscopic data such calculations can be performed, for example, in accordance with the methods described by Quesson and Kuroda (e.g. B Quesson, J A de Zwart & C T W Moonen. “Magnetic Resonance Temperature Imaging for Guidance of Thermotherapy;” 12 J Mag Res Img 525 (2000); K Kuroda, RV Mulkern, K Oshio et al. “Temperature Mapping using the Water Proton Chemical Shift; Self-referenced Method with Echo-planar Spectroscopic Imaging;” 43 Magn Reson Med 220 (2000)), both of which are incorporated herein in their entireties by reference thereto. Of course, as one skilled in the art will appreciate, other methods could also be employed. Notwithstanding which magnetic resonance parameter is used to calculate a temperature differential, the measured temperature change in a voxel will correspond to the concentration of indicator, e.g., fluid above or below body temperature, within the voxel over time.

The temperature differential determined in 40 may be used to determine a cardiovascular parameter (50). Specifically, a temperature differential can be calculated as a function of time, ΔT(t), during a dynamic acquisition. The temperature differential in a voxel of volume V corresponds to a quantity of heat, H(t), according to the formula H(t)=(ΔT(t))×(V)×(specific heat)×(specific gravity). Therefore, a cardiovascular parameter such as quantitative blood flow, F, to an individual voxel can then be determined, for example, according to the formula: (F/V)=H(t)/[AIF(t) [symbol] R(t)], where AIF(t), the arterial input function, is the quantity of heat per unit volume as a function of time at the arterial input to the voxel, R(t) is the residue function and is equal to the fraction of indicator remaining in the voxel at time t, and [symbol] denotes convolution. Such an equation can be solved using a deconvolution technique as described, for example, in L Ostergaard, R M Weisskoff, D A Chesler, C Gyldensted & B R Rosen. “High Resolution Measurement of Cerebral Blood Flow using Intravascular Tracer Bolus Passages. Part I: Mathematical Approach and Statistical Analysis.” 36 Magn Res Med 715 (1996), which is incorporated herein in its entirety by reference thereto. Alternatively, an exponential approximation can be used to calculate quantitative flow, F, for example, where the descending portion of H(t) is an exponential function such that H(t)=H₀ exp(−kt), where H₀ is the quantity of heat at time t=0 and k is a constant. By definition, k=F/V and k is then calculated based on the observed decay of H(t).

A cardiovascular parameter, such as qualitative blood flow, F, to an individual voxel can be measured, for example, according to the formula: F∝1/∫₀^{∞}H(t)dt. A cardiovascular parameter, such as mean transit time, MTT, corresponding to an individual voxel can be determined, for example, according to the formula: MTT=V/F. A cardiovascular parameter, such as volume of distribution, V, of an individual voxel can be measured, for example, according to the formula: V=(slice thickness)×(field of view)²/[(phase matrix size)×(frequency matrix size)]. Of course, other methods for determining a cardiovascular parameter will be known to one of skill in the art and the above-mentioned methods are only exemplary.

In an embodiment of a method of the present invention, a determined cardiovascular parameter can be used to produce an image in which a brightness or a color of pixels therein is determined by the cardiovascular parameter. Such an image can be produced by display systems by following methods well-known in the art, such as the method described by C Warmuth, M Gunther & C Zimmer; “Quantification of Blood Flow in Brain Tumors: Comparison of Arterial Spin Labeling and Dynamic Susceptibility weighted Contrast-enhanced MR Imaging;” 228 Radiology 523 (2003), for example, which is herein incorporated by reference thereto. For example, pixel brightness can be set equal to a linear multiple of the quantitative or the qualitative blood flow. Alternatively, pixel color can be varied to indicate higher values of flow in red and lower values of blood flow in blue on a sliding color scale.

In another embodiment, the present invention provides a machine-readable medium having stored thereon a plurality of executable instructions, which, when executed by a processor, performs obtaining magnetic resonance information from a portion of a body of a patient after introduction of fluid into a blood vessel of the patient. The plurality of executable instructions further performs determining a magnetic resonance parameter in the portion of the body using the magnetic resonance information, determining a temperature differential in the portion of the body using the magnetic resonance parameter, and determining a cardiovascular parameter using the temperature differential.

Referring to FIG. 3, the above mentioned method may be performed by a user computing device 300 such as a MRI machine, workstation, personal computer, handheld personal digital assistant (“PDA”), or any other type of microprocessor-based device. User computing device 300 may include a processor 310, input device 320, output device 330, storage device 340, client software 350, and communication device 360. Input device 320 may include a keyboard, mouse, pen-operated touch screen, voice-recognition device, or any other device that accepts input. Output device 330 may include a monitor, printer, disk drive, speakers, or any other device that provides output. Storage device 340 may include volatile and nonvolatile data storage, including one or more electrical, magnetic or optical memories such as a RAM, cache, hard drive, CD-ROM drive, tape drive or removable storage disk. Communication device 360 may include a modem, network interface card, or any other device capable of transmitting and receiving signals over a network. The components of user computing device 300 may be connected via an electrical bus or wirelessly. Client software 350 may be stored in storage device 340 and executed by processor 310, and may include, for example, imaging and analysis software that embodies the functionality of the present invention.

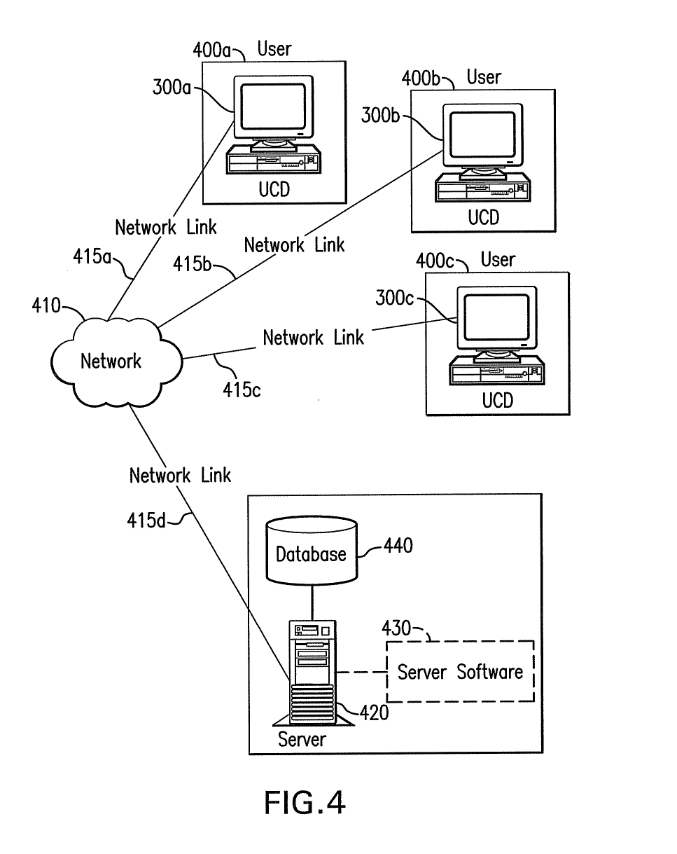

Referring to FIG. 4, the analysis functionality may be implemented on more than one user computing device 300 via a network architecture. For example, user computing device 300 may be an MRI machine that performs all determination, calculation and measurement functionality. In another embodiment, user computing device 300a may be a MRI machine that performs the magnetic resonance information measurement functionality and the magnetic resonance parameter determination functionality, and then transfers this determination over network 410 to server 420 or user computing device 300b or 300c for determination of a temperature differential and cardiovascular parameter, for example. The determined cardiovascular parameter could further be transferred to another user computing device 300 belonging to the patient or another medical services provider for further analysis.

Referring again to FIG. 4, network link 415 may include telephone lines, DSL, cable networks, T1 or T3 lines, wireless network connections, or any other arrangement that implements the transmission and reception of network signals. Network 410 may include any type of interconnected communication system, and may implement any communications protocol, which may secured by any security protocol. Server 420 includes a processor and memory for executing program instructions, as well as a network interface, and may include a collection of servers. Server 420 may include a combination of servers such as an application server and a database server. Database 440 may represent a relational or object database, and may be accessed via server 420.

User computing device 300 and server 420 may implement any operating system, such as Windows or UNIX. Client software 350 and server software 430 may be written in any programming language, such as ABAP, C, C++, Java or Visual Basic.

### Example 1

An MRI model was used to simulate flow through a capillary bed. The model included a cellulose triacetate hollow fiber dialyzer that was continuously perfused with saline at room temperature. A portion of the dialysis tubing simulated a tissue capillary bed and the continuous perfusion simulated blood flow through the cardiovascular system of the body. The model also contained a port that allowed injection of a fluid bolus into the dialysate. A power injector was utilized to inject the fluid bolus. The portion of the dialysis tubing simulating the tissue capillary bed was placed in a 1.5 T MR scanner. MR-compatible thermometers were placed proximal (thermometer 1) and distal (thermometer 2) to the simulated capillary bed with respect to the direction of flow such that fluid flowed past thermometer 1 before it flowed past thermometer 2. The port that allowed injection of the fluid bolus was placed proximal to thermometer 1 with respect to the direction of flow. A dynamic gradient echo scan was utilized to monitor the passage of the fluid bolus.

Three power-injected boluses of 30 ml of ice cold saline (4° C.) and three power-injected boluses of 60 ml of room temperature saline were administered. Prior to each injection of a fluid bolus, a baseline set of MR phase images were obtained through the simulated capillary bed and these images were used as reference image for calculation of phase changes. Additional phase images were obtained for each fluid bolus injection. The phase images were constructed on a voxel-by-voxel basis. For each fluid bolus, a temperature difference was calculated between the dynamic phase images and the reference image on a voxel-by-voxel basis using the formula

ΔT=ΔΦ(T)/αγTEB₀,

where ΔΦ(T) is the calculated phase change, α is a temperature dependent water chemical shift in ppm per C.°, γ is the gyromagnetic ratio of hydrogen, TE is the echo time and B₀ is the strength of the main magnetic field.

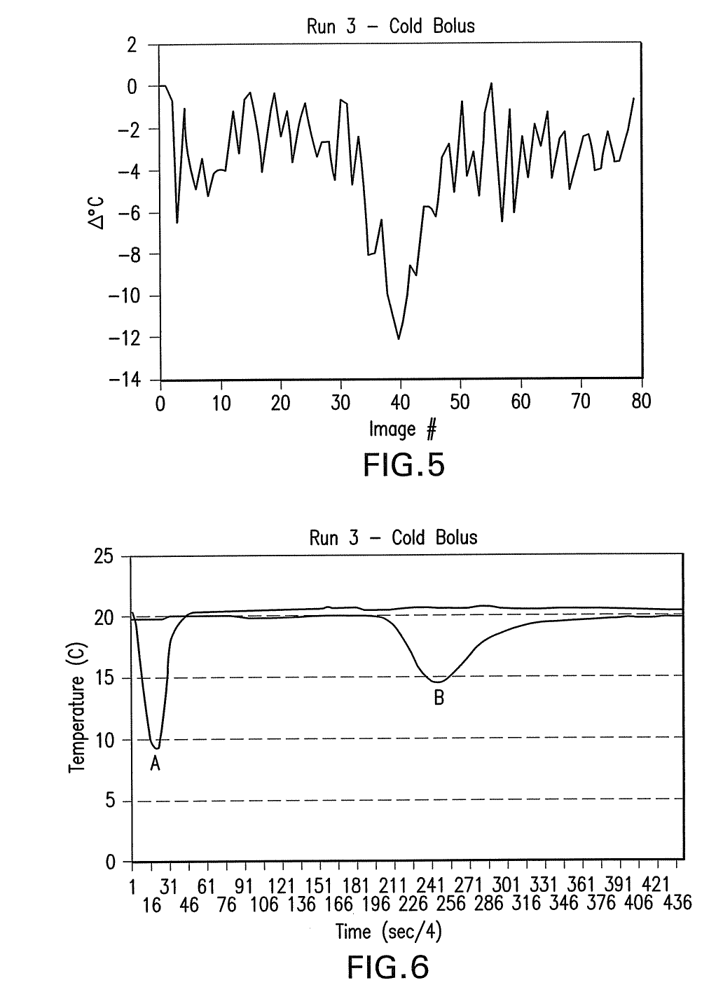

FIG. 5 is a graph of the calculated temperature differentials in sequential dynamic phase images as a function of time following an injection of a cold saline bolus. The well-defined trough in the curve corresponds to the lowest calculated temperature differential following a cold saline bolus. FIG. 6 is a graph showing the measured temperature as a function of time at thermometer 1 (A) and thermometer 2 (B) that corresponds to the cold saline bolus of FIG. 5 as the fluid bolus of cold saline passes by thermometers 1 and 2. Curve A (with the deeper trough and more narrow range) corresponds to the temperature changes over time as the fluid bolus of cold saline passes by thermometer 1 (proximal to the simulated capillary bed). Curve B (with the shallower trough and wider range) corresponds to the temperature changes over time as the fluid bolus of cold saline passes by thermometer 2 (distal to the simulated capillary bed).

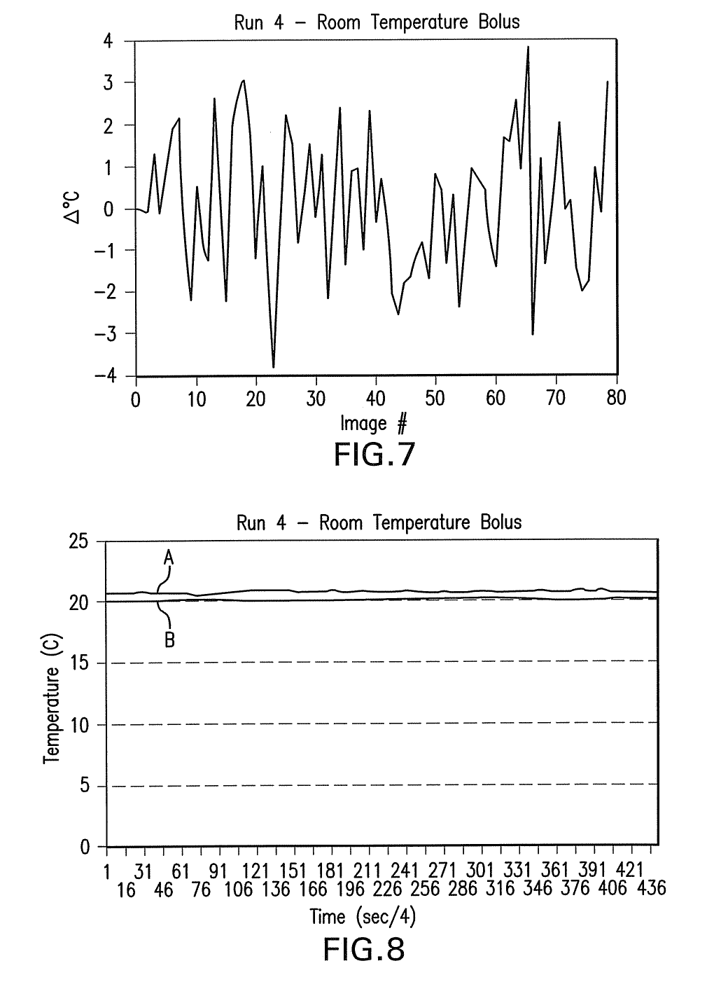

FIG. 7 is a graph of calculated temperature changes in sequential dynamic phase images as a function of time following an injection of a room temperature saline bolus. The random high frequency and low amplitude changes in the curve correspond to random fluctuations in temperature measurements due to noise. FIG. 8 is a graph showing the measured temperature as a function of time at thermometer 1 (A) and thermometer 2 (B) that corresponds to the room temperature bolus of FIG. 7. The curve remains essentially flat corresponding to no significant temperature change over time at either thermometer.

Based on this simulation model, temperature sensitive MRI measurements corresponded closely to the temperature changes detected by thermometers when a bolus of cold fluid was injected into a simulated cardiovascular system. For example, the maximal calculated decrease in temperature of FIG. 5 was approximately 12° C. and this corresponds almost exactly to the maximal decrease in temperature of curve A in FIG. 6. Furthermore, temperature sensitive MRI correctly determined that there was no temperature change when a bolus of fluid at the same temperature as the fluid in the simulated cardiovascular system was injected.

In another aspect, the present invention provides a method for determining a temperature differential in a target portion of a patient's body by using a diagnostic fluid bolus. In some cases, the diagnostic fluid bolus may be optimized for an individual patient to provide an improved determination of the temperature differential. A test fluid bolus, having a certain temperature waveform, is administered into a blood vessel, e.g., a peripheral vein, of the patient. As used herein, the term “temperature waveform” refers to the shape of the curve formed by the plot of fluid temperature versus time. The temperature waveform of the test bolus can have any of various types of waveforms. For example, the test bolus can have a step waveform, e.g., a square or rectangle. The temperature of the test bolus can be measured at any of various points prior to it being mixed with blood or body fluid, including within an apparatus delivering the fluid bolus (e.g., at convergent line temperature sensor 190 in the apparatus of FIG. 2) or within a fluid delivery device (e.g., an intravenous catheter device).

As the test bolus fluid travels to the target portion of the body, it becomes thermodiluted due to various dynamics, e.g., mixing with intravascular blood that is at body temperature. By obtaining MR information in a manner as described above, the temperature differential of the thermodiluted fluid bolus is measured in the target portion of the body. From this MR-generated information, the temperature waveform of the test fluid bolus as it arrives at the target portion of the body, i.e., the thermodiluted temperature waveform, is determined.

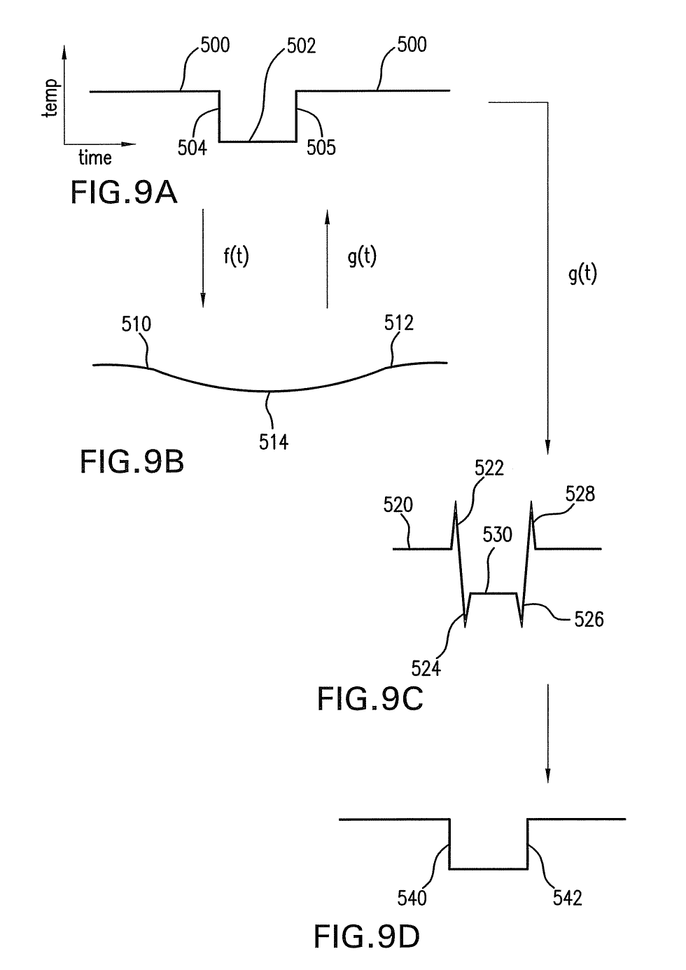

Referring to the exemplary embodiment shown in FIGS. 9A-9D, a cold test bolus is administered intravenously to a patient. FIGS. 9A-9D depict temperature (vertical axis) as a function of time (horizontal axis), with the arrows in FIG. 9A showing the direction of increasing temperature and progression of time. FIG. 9A shows the temperature waveform of the cold test bolus (as measured in the apparatus delivering the fluid). The temperature waveform of the cold test bolus has a baseline 500 (representing body-blood temperature). Administration of the cold test bolus results in a step waveform having a step-down portion 502 between leading edge 504 and trailing edge 505. The test bolus becomes thermodiluted as it travels downstream to the target site, resulting in the thermodiluted waveform 514 shown in FIG. 9B, as measured at the downstream target site. This thermodiluted waveform is blunted and has less pronounced boundaries (510 and 512) in comparison to the test bolus waveform shown in FIG. 9A.

Using the thermodiluted temperature waveform, the temperature waveform for a diagnostic fluid bolus is determined, i.e., the diagnostic bolus temperature waveform. The temperature waveform for the diagnostic fluid bolus has certain features such that it arrives at the target portion of the body with a thermodiluted temperature waveform having more pronounced boundaries, which can allow for a more accurate determination of the waveform characteristics, e.g., the area under the curve.

The diagnostic bolus temperature waveform can be determined in various ways. For example, both the test bolus waveform and its thermodiluted temperature waveform can be converted into functions using any of various curve-fitting algorithms. Subsequently, a deconvolution function that transforms the function for the thermodiluted waveform to the function for the test bolus waveform is calculated. The deconvolution function is then applied to the test bolus temperature waveform to determine a diagnostic bolus waveform for delivering the diagnostic fluid bolus. A set of parameters, e.g., fluid temperatures, fluid mixing volumes, infusion rates, etc., for delivering the diagnostic fluid bolus may then be generated. These set of parameters may then be used by an apparatus of the present invention for delivering the diagnostic fluid bolus. For example, controller 160 in the apparatus of FIG. 2 may be programmed to perform any of the various calculations or determinations described above, e.g., curve-fitting algorithms, deconvolution functions, determining diagnostic bolus parameters, determining temperature waveforms, etc.

In an exemplary embodiment, the diagnostic fluid bolus reaches the target body portion with a thermodilated waveform having more pronounced boundaries. The particular shape of the diagnostic bolus waveform will vary depending upon various factors, such as the type of fluid used, the temperature(s) of the fluid, proximity of the administration site to the target portion of the body, or patient hemodynamics.

In an exemplary embodiment, the diagnostic bolus waveform is a waveform having one or more edge-enhancing features. As used herein, the term “edge-enhancing feature” refers to a waveform feature that represents a temperature change (e.g., a temperature spike or dip) at the leading and/or trailing edge of the waveform that makes the boundaries of the thermodiluted waveform (resulting from a fluid bolus administered according to the diagnostic bolus waveform) more pronounced. The temperature change may be a departure from a baseline (e.g., body-blood temperature), a step-up portion, or step-down portion of the step waveform. The temperate change may be in either direction (hotter or colder) or both directions. In some cases, the temperature of the temperature change represented by the edge-enhancing feature is in the range of (−) 10° C. to 45° C.

The diagnostic fluid bolus and, if used, the test bolus, may have any suitable fluid volume. In some cases, the sum of the test fluid bolus volume and the diagnostic fluid bolus volume is 200 ml or less. In some cases, the volume of the diagnostic fluid bolus is greater than the volume of the test fluid bolus.

The diagnostic fluid bolus for the patient may be determined in any of various ways. In some cases, the diagnostic fluid bolus may be determined using a test fluid bolus in the manner described above. In some cases, the diagnostic fluid bolus may be selected on the basis of a physiologic measurement or medical condition of the patient. Examples of physiologic measurements include hemodynamic measurements (e.g., cardiac ejection fraction, heart rate, heart rhythm, pulmonary arterial blood pressure, or systemic blood pressure), respiratory rate, respiratory cycle, respiratory rhythm, pulse oximeter, body temperature, and blood count measurements (e.g., hematocrit or hemoglobin). Examples of medical conditions include heart failure, hypertension, sepsis, hypovolemia due to blood loss, etc.

In some cases, the user may selected a diagnostic fluid bolus having a temperature waveform with one or more edge-enhancing features. For example, the diagnostic fluid bolus may not be optimized for an individual patient, but is nonetheless capable of providing a thermodiluted temperature waveform having more pronounced boundaries.

The diagnostic fluid bolus may also be selected using a database containing relational associations between: (a) a plurality of physiologic measurements or medical conditions; and (b) a plurality of diagnostic bolus temperature waveforms and/or a plurality of set of parameters for delivering a diagnostic fluid bolus. This database can be created using data collected from performing the above-described method for determining a diagnostic fluid bolus (using a test fluid bolus) on multiple patients having various physiologic measurements and/or medical conditions. Mathematical modeling may be applied to the data to assist in making the correlations needed for the relational associations.

The temperature differential can be measured using any suitable MR-based method, including those described above, or any other conventional method for determining a temperature differential e.g., by catheters and/or thermometers. The above-described steps for determining a temperature differential using a diagnostic fluid bolus and/or a test fluid bolus can be performed by an apparatus of the present invention. Also, various of the above-described steps (including the determinations, calculations, obtaining of MR information, obtaining of temperature information, etc.) may be performed by software.

In the exemplary embodiment of FIGS. 9A-9D, information regarding the test bolus waveform of FIG. 9A and the thermodilution waveform of FIG. 9B is used to determine the parameters for delivering a diagnostic fluid bolus that would lead to a thermodiluted waveform having more pronounced boundaries, e.g., having more resemblance to the temperature waveform of the test bolus (as measured in the apparatus delivering the fluid), when it arrives at the target site. This is achieved by determining the deconvolution function g(t) that transforms the thermodiluted waveform of FIG. 9B to the test bolus waveform of FIG. 9A. Alternatively, one could determine the convolution function ƒ(t) that transforms the test bolus waveform of FIG. 9A to the thermodiluted waveform of FIG. 9B. The deconvolution function g(t) is the inverse mathematical operation of the convolution function ƒ(t). The deconvolution function and/or convolution function may employ any of various types of mathematical operations for transforming one function into another function e.g., Fourier transformations.

The deconvolution function g(t) is then applied to the test bolus waveform shown in FIG. 9A. This may result in a diagnostic bolus temperature waveform as shown in FIG. 9C. The diagnostic bolus temperature waveform illustrated in FIG. 9C is a step waveform having a baseline 520 (representing body-blood temperature); a step-down portion 530; and edge-enhancing features 522, 524, 526, and 528. The diagnostic bolus waveform shown in FIG. 9C is only representative of this particular example, and other diagnostic bolus waveforms are possible. For example, in the step waveform of FIG. 9C, various other combinations of the edge-enhancing features are possible, e.g., having just edge-enhancing features 522 and 528, or having just edge-enhancing features 524 and 526. Furthermore, the diagnostic bolus temperature waveforms may not necessarily have edge-enhancing features or may not necessarily be a step waveform. The diagnostic bolus temperature waveforms may have other types of features that would lead to a thermodiluted waveform having more pronounced boundaries. These features may vary but, for example, may include increasing the area under the waveform curve, increasing a step-up or step-down portion of the waveform, increasing the slope of the leading edge and/or trailing edge, or making the waveform narrower.

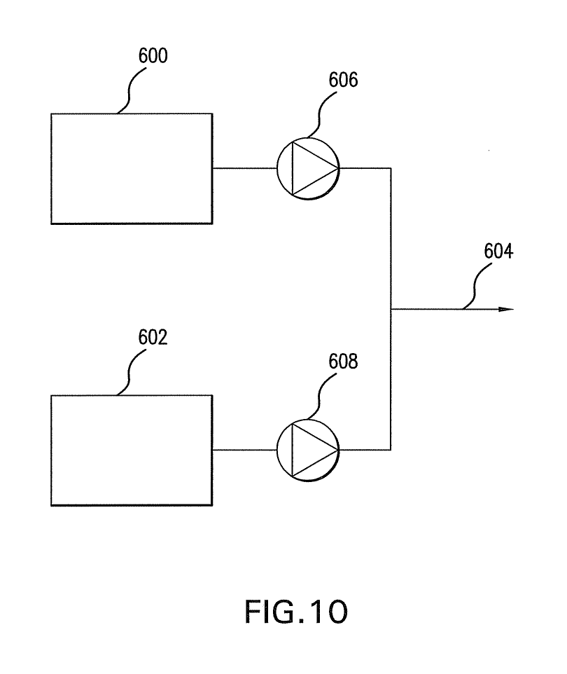

FIG. 10 shows an apparatus, according to an embodiment, for delivering a diagnostic fluid bolus having the diagnostic bolus temperature waveform of FIG. 9C. The apparatus includes a reservoir 600 containing heated fluid, e.g., at 42° C., and a reservoir 602 containing cooled fluid, e.g., at −5° C. Reservoir 600 is connected to a valve 606, which controls the flow of the heated fluid in reservoir 600 into a catheter device 604 that is inserted into a patient. Reservoir 602 is connected to a valve 608, which controls the flow of the cooled fluid in reservoir 602 into the catheter device 604. Valves 606 and 608 may be controlled manually or by a computer (e.g., by controller 160 in FIG. 2) to deliver fluid through catheter device 604 having a desired temperature. The apparatus may also include one or more pumps for controlling the flow of fluid. The temperature of the fluid bolus can be measured at the catheter device 604 or at a convergent line that receives the fluid from both reservoir 600 and reservoir 602.

A set of parameters for the opening and/or closing of valves 606 and 608 are determined (e.g., by controller 160 in FIG. 2) for delivering an diagnostic fluid bolus having the diagnostic bolus waveform shown in FIG. 9C. For example, the parameters may dictate a short burst of the heated fluid for a temperature spike representing edge-enhancing feature 522 of the diagnostic bolus waveform; followed by a short burst of the cooled fluid for a temperature dip representing edge-enhancing feature 524; followed by a constant infusion of fluid at an intermediate temperature, e.g., 4° C., representing step-down portion 530; followed by a short burst of the cooled fluid for a temperature dip representing edge-enhancing feature 526; and followed by a short burst of the heated fluid for a temperature spike representing edge-enhancing feature 528.

The diagnostic fluid bolus becomes thermodiluted as it travels downstream to the target site, resulting in the thermodiluted waveform shown in FIG. 9D, as measured at the downstream target site. This thermodiluted waveform has more pronounced boundaries (540 and 542) in comparison to the thermodiluted waveform shown in FIG. 9B. These more pronounced boundaries are the result of the edge-enhancing features in the diagnostic bolus waveform of FIG. 9C.

The foregoing description and example have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A method for determining a temperature differential in a portion of a patient's body, comprising:
administering a diagnostic fluid bolus into a blood vessel of the patient, wherein the temperature waveform of the diagnostic fluid bolus is at least one of:
i) a waveform having an edge-enhancing feature; and
ii) selected using information obtained from a test fluid bolus administered into the blood vessel after the test fluid bolus has traveled a distance to the portion of the body;
obtaining a diagnostic set of magnetic resonance information from the portion of the body;
determining a magnetic resonance parameter from the portion of the body using the diagnostic set of magnetic resonance information; and
determining a temperature differential in the portion of the body using the magnetic resonance parameter.

2. The method of claim 1, wherein the diagnostic fluid bolus is selected by administering the test fluid bolus, and wherein the method further comprises:
administering the test fluid bolus into the blood vessel of the patient, wherein the test fluid bolus has a temperature waveform;
obtaining a test set of magnetic resonance information from the portion of the body; and
using the test set of magnetic resonance information to determine a thermodiluted temperature waveform that results from thermodilution of the test fluid bolus as the test fluid bolus travels to the portion of the body.

3. The method of claim 2, further comprising:
determining a deconvolution function that transforms the thermodiluted temperature waveform into the temperature waveform of the test fluid bolus; and
applying the deconvolution function to the temperature waveform of the test fluid bolus to obtain the temperature waveform for the diagnostic fluid bolus.

4. The method of claim 3, further comprising determining a convolution function that transforms the test fluid bolus temperature waveform into the thermodiluted temperature waveform.

5. The method of claim 4, further comprising obtaining the deconvolution function based on the convolution function.

6. The method of claim 5, wherein the deconvolution function is the inverse mathematical operation of the convolution function.

7. The method of claim 3, wherein the deconvolution function employs a Fourier transformation.

8. The method of claim 4, wherein the convolution function employs a Fourier transformation.

9. The method of claim 2, wherein the sum of the volume of the test fluid bolus and the volume of the diagnostic fluid bolus is 250 ml or less.

10. The method of claim 9, wherein the volume of the diagnostic fluid bolus is greater than the volume of the test fluid bolus.

11. The method of claim 1, wherein the portion of the body is an organ.

12. The method of claim 11, wherein the organ is the brain.

13. The method of claim 1, wherein the temperature waveform of the diagnostic fluid bolus is a waveform having an edge-enhancing feature selected from a plurality of pre-recorded waveforms, and wherein the selection is made on the basis of a physiologic measurement or medical condition of the patient.

14. The method of claim 13, wherein the physiologic measurement is a hemodynamic measurement or a blood count measurement.

15. The method of claim 13, wherein the physiologic measurement is one of cardiac ejection fraction, heart rate, heart rhythm, pulmonary arterial blood pressure, respiratory rate, respiratory cycle, respiratory rhythm, pulse oximeter, body temperature, systemic blood pressure, hematocrit, and hemoglobin.

16. The method of claim 13, wherein the selection of the temperature waveform for the diagnostic fluid bolus comprises using a database containing relational associations between: (a) a plurality of physiologic measurements or medical conditions; and (b) a plurality of pre-recorded waveforms or a plurality of set of parameters for delivering a fluid bolus according to a pre-recoded waveform.

17. The method of claim 16, wherein the database is created by using thermodiluted temperature waveform data obtained from administering test fluid boluses to a plurality of patients and correlating the thermodiluted temperature waveform data to physiologic measurements or medical conditions of the plurality of patients.

18. The method of claim 1, wherein the temperature waveform of the diagnostic fluid bolus is a step waveform having the edge-enhanced feature.

19. The method of claim 18, wherein the edge-enhancing feature represents a temperature change from at least one of a baseline of the step waveform, a step-up portion of the step waveform, and a step-down portion of the step waveform.

20. The method of claim 1, wherein the temperature represented by the edge-enhancing feature is in the range of (−)10° C. to 45° C.

21. The method of claim 1, wherein the temperature waveform of the diagnostic fluid bolus has an edge-enhancing feature at both the leading edge and the trailing edge.

22. The method of claim 1, wherein the temperature waveform of the diagnostic fluid bolus has two or more edge-enhancing features.

23. An apparatus for determining a temperature differential in a portion of a patient's body, the apparatus comprising:
a fluid source;
a fluid control system adapted to control flow of the fluid from the fluid source into the patient; and
a controller in communication with the fluid control system and adapted to:
operate the fluid control system to deliver a diagnostic fluid bolus to be administered into a blood vessel of the patient, wherein the temperature waveform of the diagnostic fluid bolus is at least one of:
i) a waveform having an edge-enhancing feature; and
ii) selected using information obtained from a test fluid bolus administered into the blood vessel after the test fluid bolus has traveled a distance to the portion of the body;
obtain a diagnostic set of magnetic resonance information from a portion of the patient's body after administration of the diagnostic fluid bolus;
determine a magnetic resonance parameter from the portion of the body using the diagnostic set magnetic resonance information; and
determine a temperature differential in the portion of the body using the magnetic resonance parameter.

24. The apparatus of claim 23, wherein the controller is programmed to further perform the following:
operate the fluid control system to deliver a test fluid bolus to be administered into the blood vessel of the patient;
obtain a test set of magnetic resonance information from the portion of the body after the administration of a test fluid bolus into the blood vessel of the patient;
use the test set of magnetic resonance information to determine a thermodiluted temperature waveform that results from thermodilution of the test fluid bolus as the test fluid bolus travels to the portion of the body;
determine a deconvolution function that transforms the thermodiluted temperature waveform into the temperature waveform of the test fluid bolus; and
apply the deconvolution function to the temperature waveform of the test fluid bolus to obtain a temperature waveform for the diagnostic fluid bolus.

25. The apparatus of claim 23, wherein the controller is programmed to further perform the following:
receive a physiologic measurement or medical condition of the patient;
select the temperature waveform of the diagnostic fluid bolus from a plurality of pre-recorded waveforms, and wherein the selection is made on the basis of the received physiologic measurement or medical condition of the patient.

26. The apparatus of claim 25, wherein the physiologic measurement is a hemodynamic measurement or a blood count measurement.

27. The apparatus of claim 25, wherein the physiologic measurement is one of cardiac ejection fraction, heart rate, heart rhythm, pulmonary arterial blood pressure, respiratory rate, respiratory cycle, respiratory rhythm, pulse oximeter, body temperature, systemic blood pressure, hematocrit, and hemoglobin.

28. The apparatus of claim 25, wherein the selection of the temperature waveform for the diagnostic fluid bolus comprises using a database containing relational associations between: (a) a plurality of physiologic measurements or medical conditions; and (b) a plurality of pre-recorded waveforms or a plurality of set of parameters for delivering a fluid bolus according to a pre-recoded waveform.

29. The apparatus of claim 24, wherein the fluid source comprises a first fluid at a first temperature and a second fluid at a second temperature, and wherein the fluid control system is adapted to mix the first fluid and the second fluid to deliver a mixed fluid having a third temperature into the patient.

30. The apparatus of claim 29, wherein the third temperature is in the range of (−)10° C. to 45° C.

31. The apparatus of claim 23, further comprising a magnetic resonance scanner.
